(19) United States
(12) Patent Application Publication
(10) Pub. No.: US 2021/0113854 A1
(43) Pub. Date: Apr. 22, 2021

(54) LASER LIPOLYSIS APPARATUS AND LASER LIPOLYSIS PROCEDURE METHOD

(71) Applicant: LUTRONIC CORPORATION, Goyang (KR)

(72) Inventors: Hyun Min SHIN, Seoul (KR); Hae Lyung HWANG, Goyang (KR)

(21) Appl. No.: 16/608,605

(22) PCT Filed: Apr. 20, 2018
(86) PCT No.: PCT/KR2018/004603

§ 371 Date: Oct. 25, 2019

(30) Foreign Application Priority Data
Apr. 28, 2017 (KR) 10-2017-0055175

(51) Int. Cl. A61N 5/06

(52) U.S. Cl. CPC A61N 5/0625; A61N 2005/067

(57) ABSTRACT

A laser lipolysis apparatus according to an embodiment of the present invention comprises: a first light irradiation unit for emitting a laser of a first wavelength reaching an upper region of a subcutaneous fat layer; and a second light irradiation unit for emitting a laser of a second wavelength reaching a lower region of the subcutaneous fat layer.

### TECHNICAL FIELD

The present invention relates to a laser lipolysis apparatus and a laser lipolysis procedure method, and more particularly to a laser lipolysis apparatus and a laser lipolysis procedure method for breaking down the subcutaneous fat layer by use of a laser.

### BACKGROUND ART

In general, human skin consists of the epidermis (epithelium), the subcutaneous fat layer positioned under the epidermis, and the dermis positioned between the epidermis and the subcutaneous fat layer. The epidermis is a skin surface exposed outside, and the epidermis is composed of matrix proteins such as collagen fibers and elastic fibers and contains blood vessels, nerves, sweat glands, etc. The subcutaneous fat layer is positioned under the skin surface and composed of fat tissues in order to protect from heat damage and act as a shock absorber and a nutrition storage.

Obesity is caused by various factors, mainly excessive fat accumulation in a human body. For example, obesity is caused by increase in the number or size of fat tissues in the subcutaneous fat layer due to excessive fat accumulation. Such obesity may lead the human body to extend outward in a local area of skin, which degrades aesthetic properties.

In order to address the aesthetically interruptive factor, an invasive method and a non-invasive method for reducing the subcutaneous layer or fat tissues have been introduced. As the non-invasive method, there is a method of reducing the subcutaneous fat layer by applying heat to the subcutaneous fat layer by use of a high frequency or a laser beam.

### DISCLOSURE

### Technical Problem

The present invention provides a laser lipolysis apparatus and a laser lipolysis procedure method for effectively breaking down subcutaneous fat.

Objects of the present invention are not limited to those described above and other unmentioned objects will be more clearly understood by a person skilled in the art from the following description

### Technical Solution

In one aspect of the present invention a laser lipolysis apparatus includes a first light irradiation unit for emitting a laser of a first wavelength reaching an upper region of a subcutaneous fat layer; and a second light irradiation unit for emitting a laser of a second wavelength reaching a lower region of the subcutaneous fat layer.

The laser of the first wavelength and the laser of the second wavelength may be irradiated toward a skin surface such that at least some of the lasers penetrate epidermis and dermis and reach the subcutaneous fat layer.

The first wavelength may be shorter than the second wavelength.

The first wavelength may be 635 nm and the second wavelength is 1064 nm.

The laser of the first wavelength and the laser of the second wavelength may be emitted alternately.

The laser lipolysis apparatus may further include a first hand piece provided with the first light irradiation unit, a second hand piece provided with the second light irradiation unit.

The laser lipolysis apparatus may further include a hand piece provided with the first light irradiation unit and the second light irradiation unit.

The laser lipolysis apparatus may further include a fixed member for fixing at least one of the first light irradiation unit or the second light irradiation unit to a body of a patient.

The first light irradiation unit and the second light irradiation unit may be detachably installed in the fixed member.

The fixed member may be brought into contact tightly with the body of the patient while surrounding at least one of abdomen, a leg, or an arm of the patient.

In another aspect of the present invention, a laser lipolysis procedure method including irradiating skin of a patient with a laser of a first wavelength reaching an upper region of a subcutaneous fat layer, and irradiating the skin of the patient with a laser of a second wavelength reaching a lower region of the subcutaneous fat layer.

The first wavelength may be shorter than the second wavelength.

The first wavelength may be selected within a range equal to or greater than 630 nm and equal to or smaller than 640 nm, and the second wavelength is selected within a range equal to or greater than 1060 nm and equal to or smaller than 1070 nm.

The laser of the first wavelength and the laser of the second wavelength may be irradiated alternately.

Other concrete matters of the present invention are included in the detailed description and drawings.

### Advantageous Effects

According to embodiments of the present invention, there are effects at least as follows.

It is possible to more effectively break down the subcutaneous fat layer.

Advantageous effects according to the present invention are not limited to those mentioned above, and various other advantageous effects are included herein.

### DESCRIPTION OF DRAWINGS

FIG. 1 is a diagram for explaining a laser lipolysis procedure method according to an embodiment of the present invention.

FIG. 2 is a diagram schematically illustrating a laser lipolysis apparatus according to a first embodiment of the present invention.

FIG. 3 is a diagram schematically illustrating a laser lipolysis apparatus according to a second embodiment of the present invention.

FIG. 4 is a perspective view illustrating a fixed member of the laser lipolysis apparatus according to the second embodiment of the present invention.

FIG. 5 is a diagram illustrating an example of use of the laser lipolysis apparatus according to the second embodiment of the present invention.

FIG. 6 is a diagram schematically illustrating a laser lipolysis apparatus according to a third embodiment of the present invention.

FIG. 7 is a diagram schematically illustrating a front end of a hand piece of FIG. 6.

FIG. 8 is a diagram schematically illustrating a laser lipolysis apparatus according to a fourth embodiment of the present invention.

FIG. 9 is a diagram schematically illustrating a front end of a hand piece of a laser lipolysis apparatus according to a fifth embodiment of the present invention.

FIG. 10 is a diagram schematically illustrating a front end of a hand piece of a laser lipolysis apparatus according to a sixth embodiment of the present invention.

### BEST MODE

The advantages and features of the present invention and methods of achieving them will be apparent from the following exemplary embodiments that will be described in more detail with reference to the accompanying drawings. It should be noted, however, that the present invention is not limited to the following exemplary embodiments and may be implemented in various forms, and accordingly, the exemplary embodiments are provided only to disclose the present invention and let those skilled in the art know the category of the present invention. In the drawings, embodiments of the present invention are not limited to the specific examples provided herein and are exaggerated for clarity. The same reference numerals or the same reference designators denote the same elements throughout the specification.

In addition, embodiments herein will be described with reference to cross-sectional views and/or schematic views, which are ideal exemplary views of the present invention. Therefore, the form of an exemplary view may be deformed due to a manufacturing technique and/or an allowable error. In addition, in each drawing of the present disclosure, each element may have been somewhat enlarged or reduced in consideration of convenience of description. Like reference numerals refer to like elements throughout.

Hereinafter, the present invention will be described in detail with reference to the accompanying drawings for explaining a laser lipolysis apparatus and a laser lipolysis procedure method according to an embodiment of the present invention.

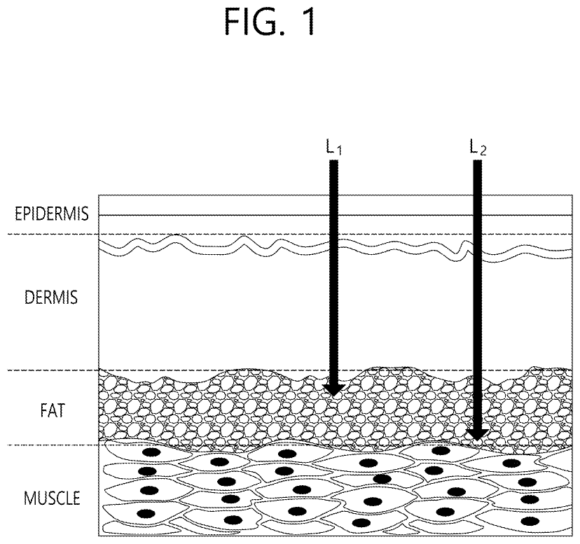

FIG. 1 is a diagram for explaining a laser lipolysis procedure method according to an embodiment of the present invention.

As illustrated in FIG. 1, laser lipolysis procedure method according to an embodiment of the present invention is performed by irradiating two lasers L1 and L2 of different wavelengths.

The laser L1 of a first wavelength and the laser L2 of a second wavelength are irradiated from an outside of skin of a patient.

The laser L1 of the first wavelength and the laser L2 of the second wavelength have to reach to the subcutaneous fat layer and have to be lasers of wavelengths which are excellent in fat absorption for effective lipolysis.

To this end, the laser lipolysis method and the laser lipolysis apparatus according to an embodiment of the present invention employ a laser of 635 nm wavelength as the first laser L1 of the first wavelength and a laser of 1064 nm wavelength as the second laser L2 of the second wavelength. The laser L1 of 635 nm wavelength may be an ALGaINP semi-conductor laser diode, and the laser L2 of 1064 nm wavelength may be an NdYAG laser diode or an AI GaAs semi-conductor laser diode.

As illustrated in FIG. 1, the laser L1 of the first wavelength reaches an upper region of the subcutaneous fat layer, and the layer L2 of the second wavelength may reach a lower region of the subcutaneous fat layer.

When the layer L1 of the first wavelength and the laser L2 of the second wavelength are irradiated for a predetermined period of time, the upper region of the subcutaneous fat layer absorbs primarily the laser L1 of the first wavelength and is thereby gradually broken down, and the lower region of the subcutaneous fat layer absorbs primarily the laser L2 of the second wavelength and is thereby gradually broken down.

The laser lipolysis method according to an embodiment of the present invention may be able to break down fat, as compared to an existing method of breaking down fat with a laser of a single wavelength, since the two lasers L1 and L2 of different wavelengths break down fat tissues in the upper and lower regions of the subcutaneous fat layer.

The laser L1 of the first wavelength and the laser L2 of the second wavelength may be irradiated simultaneously and may be irradiated alternately.

Hereinafter, a laser lipolysis apparatus according to various embodiments, which is used in the above-described laser lipolysis procedure method, will be described.

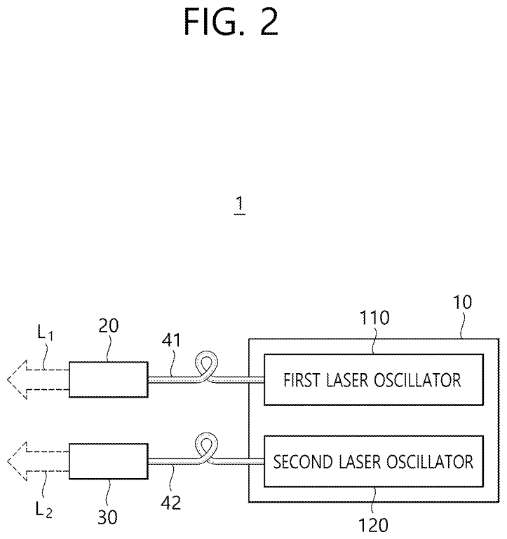

FIG. 2 is a diagram schematically illustrating a laser lipolysis apparatus according to a first embodiment of the present invention.

As illustrated in FIG. 2, a laser lipolysis apparatus 1 according to a first embodiment of the present invention includes a main body 10, a first hand piece 20, and a second hand piece 30.

The main body 10 includes a first laser oscillator 110 and a second laser oscillator 120. The first laser oscillator 110 generates a laser L1 of a first wavelength, and the second laser oscillator 120 generates a laser L2 of a second wavelength.

The first hand piece 20 is connected with the first laser oscillator 110 through an optical cable 41. Accordingly, the laser L1 of the first wavelength generated in the first laser oscillator 110 is delivered to the first hand piece 20 through the optical cable 41.

The first hand piece 20 serves as a first light irradiation unit that emits the laser L1 of the first wavelength, delivered through the optical cable 41, toward skin of a patient. The first hand piece 20 may include at least one optical mechanism that diverges or converges the laser L1 of the first wavelength, delivered through the optical cable 41, into a predetermined beam pattern.

The second hand piece 30 is connected with the second layer oscillator 120 through an optical cable 42. Accordingly, the laser L2 of the second wavelength, generated in the second laser oscillator 120, is delivered to the second hand piece 30 through the optical cable 42.

The second hand piece 30 serves as a second light irradiation unit that emits the laser L2 of the second wavelength, delivered through the optical cable 42, toward skin of a patient. The second hand piece 30 may include at least one optical mechanism that diverges or converges the laser L2 of the second wavelength, delivered through the optical cable 42, into a predetermined beam pattern.

Although not illustrated, the main body 10 of the laser lipolysis apparatus 1 according to the present embodiment may further include a controller that controls the first laser oscillator 110 and the second laser oscillator 120.

According to an embodiment, the controller may simultaneously drive the first laser oscillator 110 and the second laser oscillator 120 so as to simultaneously emit the laser L1 of the first wavelength and the laser L2 of the second wavelength, or may alternately drive the first laser oscillator 110 and the second laser oscillator 120 so as to alternately emit the laser L1 of the first wavelength and the laser L2 of the second wavelength.

Hereinafter, a laser lipolysis apparatus according to another embodiment will be described. For convenience of explanation, the same reference numerals are used for elements similar to those from the above-described first embodiment, and descriptions of elements that the present exemplary embodiment has in common with the above-described first embodiment will be omitted.

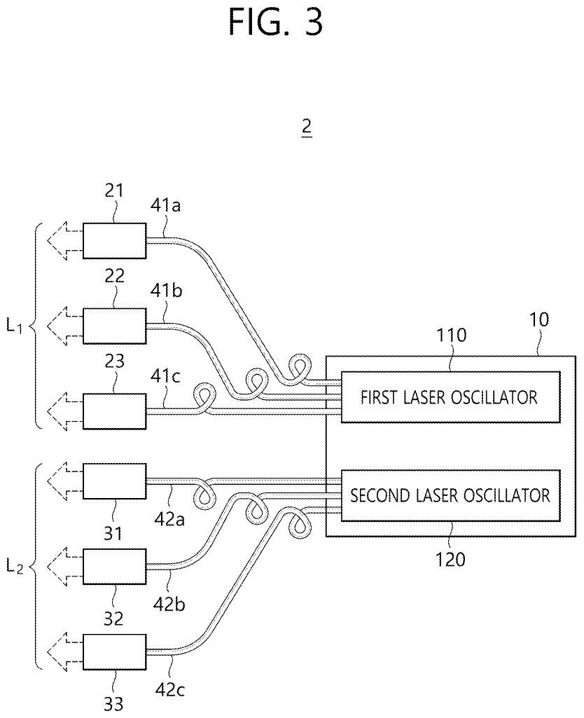

FIG. 3 is a diagram schematically illustrating a laser lipolysis apparatus according to a second embodiment of the present invention.

As illustrated in FIG. 3, as compared with the laser lipolysis apparatus 1 according to the above-described first embodiment, a laser lipolysis apparatus 2 according to a second embodiment of the present invention is configured such that a first light irradiation unit for irradiating a laser L1 of a first wavelength includes a plurality of hand pieces 21, 22, and 23 and similarly a second light irradiation unit for emitting a laser L1 of a second wavelength includes a plurality of second hand pieces 31, 32, and 33.

As illustrated in FIG. 3, the plurality of first hand pieces 21, 22, and 23 may be connected with a first laser oscillator 110 individually through a plurality of optical cables 41a, 41b, and 41c, and similarly the plurality of second hand pieces 31, 32, and 33 may be connected with the second individual 120 individually through a plurality of optical cables 42a, 42b, and 42c.

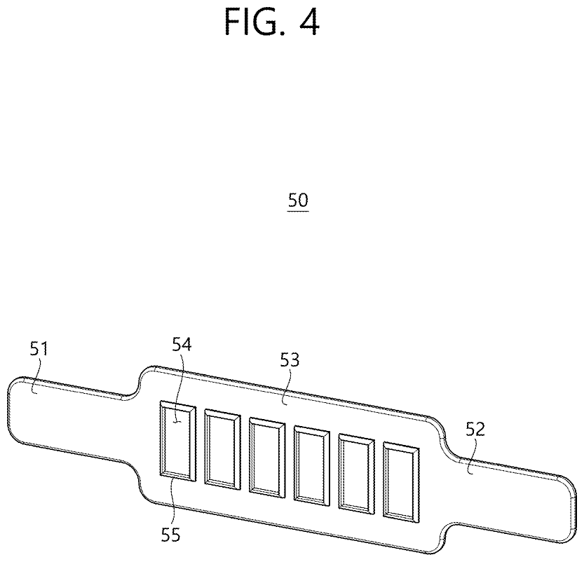

FIG. 4 is a perspective view illustrating a fixed member of a laser lipolysis apparatus according to a second embodiment of the present invention.

As illustrated in FIG. 4, a laser lipolysis apparatus 2 according to the present embodiment further includes a fixed member 50.

The fixed member 50 includes a mounting portion 53 with a plurality of hand piece mounting holes 54, a first coupling portion 51 extending from the mounting portion 53, and a second coupling portion 52 extending from the mounting portion 53 toward the other side.

The plurality of hand piece mounting portions 54 is formed to penetrate the fixed member 50, and a hand piece supporting end 55 provided to surround each hand piece mounting hole 54 is formed in the mounting portion 53 to protrude.

The plurality of hand piece mounting holes 54 is spaces into which at least some of the plurality of first hand pieces 21, 22, and 23 and the plurality of second hand pieces 31, 32, and 33, and the hand piece supporting end 55 is an element that supports and fixes the first hand pieces 21, 22, and 23 or the second hand pieces 31, 32, and 33 inserted into the respective hand piece mounting holes 54.

The plurality of first hand pieces 21, 22, and 23 and the plurality of second hand pieces 31, 32, and 33 are detachably coupled to the hand piece mounting holes 54 and the hand piece supporting ends 55.

The fixed member 50 is used in a manner in which the mounting portion 53 is brought into contact tightly with abdomen, an arm, or a leg of a patient, similarly to the case of fastening a belt, and the first coupling portion 51 and the second coupling portion 52 is fixed to each other.

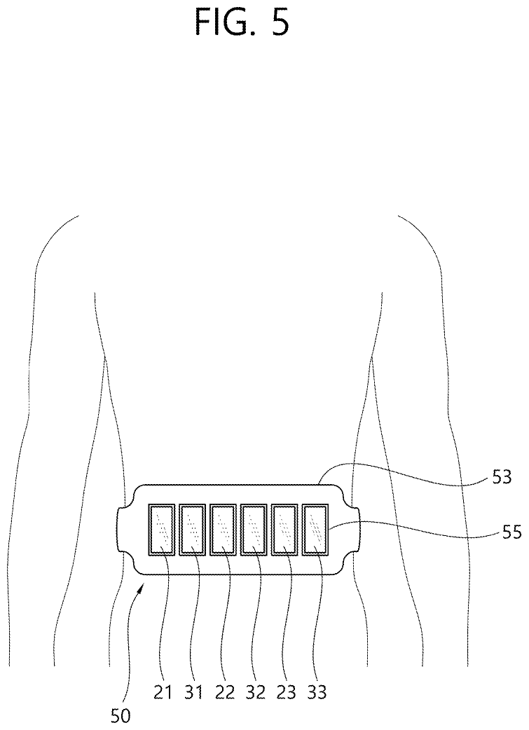

FIG. 5 is a diagram illustrating an example of use of the laser lipolysis apparatus according to the second embodiment of the present invention.

As illustrated in FIG. 5, the laser lipolysis apparatus according to the second embodiment of the present invention may be used in a manner in which the fixed member 50 is put on over the waist of a patient, like a belt, and the plurality of first hand pieces 21, 22, and 23 and the plurality of second hand pieces 31, 32, and 33 are inserted into the respective hand piece mounting holes 54 and thereby fixed thereto.

As illustrated in FIG. 5, the plurality of first hand pieces 21, 22, and 23 and the plurality of second hand pieces 31, 32, and 33 may be alternately mounted on the plurality of hand piece mounting holes 54. When the plurality of first hand pieces 21, 22, and 23 and the plurality of second hand pieces 31, 32, and 33 are alternately mounted, the laser L1 of the first wavelength and the laser L2 of the second wavelength may be generally equally irradiated over the subcutaneous fat layer in the abdomen.

However, according to an embodiment, the arrangement relationship between the plurality of first hand pieces 21, 22, and 23 and the plurality of second hand pieces 31, 32, and 33 may change.

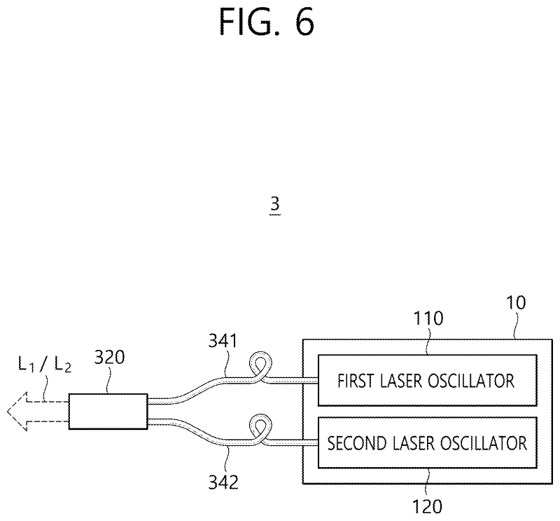

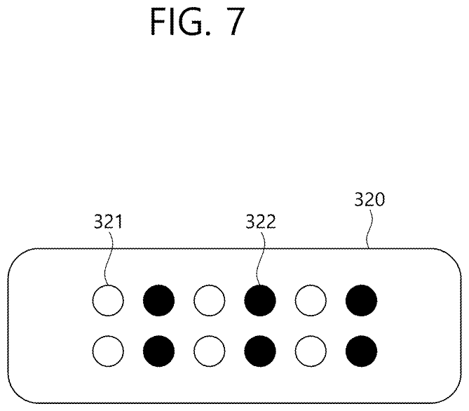

FIG. 6 is a diagram schematically illustrating a laser lipolysis apparatus according to a third embodiment of the present invention, and FIG. 7 is a diagram schematically illustrating a front end of a hand piece of FIG. 6.

As illustrated in FIG. 6, as compared with the laser lipolysis apparatus 1 according to the above-described first embodiment, a laser lipolysis apparatus 3 according to a third embodiment of the present invention is configured such that a first light irradiation unit and a second light irradiation unit are provided in a single hand piece 320.

That is, a laser L1 of a first wavelength and a laser L2 of a second wavelength are emitted from the single hand piece 320.

The hand piece 320 are connected with a first laser oscillator and a second laser oscillator 123 through optical cables 341 and 342 so that the first laser L1 of the first wavelength and the laser L2 of the second wavelength are delivered respectively from the first laser oscillator 110 and the second laser oscillator 120 and then emitted to an outside.

As illustrated in FIG. 7, a first light irradiation unit 321 for emitting the laser L1 of the first wavelength and a second light irradiation unit 322 for emitting the laser L2 of the second wavelength may exist together at a front end of the hand piece 320.

As illustrated in FIG. 7, the first light irradiation unit 321 and the second light irradiation unit 322 may be uniformly distributed at the front end of the hand piece 320. Alternatively, according to an embodiment, the first light irradiation unit 321 and the second light irradiation unit 322 may be distributed in separate regions.

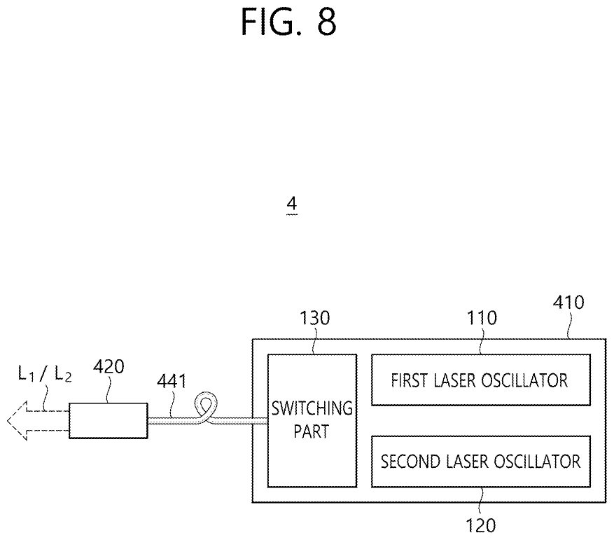

FIG. 8 is a diagram schematically illustrating a laser lipolysis apparatus according to a fourth embodiment of the present invention.

As illustrated in FIG. 8, as compared with the laser lipolysis apparatus 3 according to the above-described third embodiment, a laser lipolysis apparatus 4 according to a fourth embodiment of the present invention is configured such that a switching part 130 is further included in a main body 410 and an optical cable connected with a hand piece 420 is connected with the switching part 130.

The switching part 130 is connected with both a first laser oscillator 110 and a second laser oscillator 120 and thus receives a laser L1 of a first wavelength and a laser L2 of a second wavelength.

In accordance with a control of a controller (not shown), the switching part 130 delivers any one of the delivered laser L1 of the first wavelength and the delivered laser L2 of the second wavelength to the optical cable 441.

Accordingly, the hand piece 420 does not simultaneously emit the laser L1 of the first wavelength and the laser L2 of the second wavelength but selectively emit any one of the laser L1 of the first wavelength and the laser L2 of the second wavelength.

That is, in a case where the switching part 130 delivers the laser L1 of the first wavelength to the optical cable 441, the laser L1 of the first wavelength is emitted through the hand piece 420, and, in a case where the switching part 1340 delivers the laser L2 of the second wavelength to the optical cable 441, the laser L2 of the second wavelength is emitted through the hand piece 420.

Therefore, a user may control the laser L1 of the first wavelength and the laser L2 of the second wavelength to be alternately emitted through the hand piece 420 or may control only one of the laser L1 of the first wavelength and the laser L2 of the second wavelength to be constantly emitted through the hand piece 420.

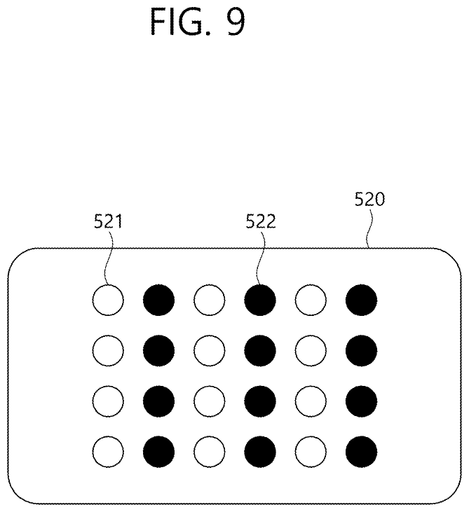

FIG. 9 is a diagram schematically illustrating a front end of a hand piece of a laser lipolysis apparatus according to a fifth embodiment of the present invention.

Unlike the laser lipolysis apparatuses 1, 2, 3, and 4 according to the above-described embodiments, the laser lipolysis apparatus according to the fifth embodiment of the present invention is provided with a plurality of laser diodes 521 and 522, instead of adapting a technique in which a hand piece receives a laser from a main body and irradiates the received laser.

Accordingly, the lipolysis apparatus according to the present embodiment does not include a first laser oscillator 110 and a second laser oscillator 120 and does not use an optical cable that connects a main body and a hand piece. The hand piece 520 is provided from the main body with electric power required for light emission of the plurality of laser diodes 521 and 522.

In the present embodiment, a first light irradiation unit for emitting a laser L1 of a first wavelength includes first laser diodes 521, and a second light irradiation unit for emitting a laser L2 of a second wavelength includes second laser diodes 522.

As illustrated in FIG. 9, the first laser diodes 521 and the second laser diodes 522 may be arranged such that light are uniformly distributed over a front surface of the hand piece 520.

The first laser diodes 521 and the second laser diodes 522 may be turned on simultaneously and may be simultaneously or alternately turned on in a pre-programmed order and time according to an embodiment.

Or, similarly to the laser lipolysis apparatus according to the first embodiment or the second embodiment, two or more hand pieces may be included such that the first laser diodes 521 may be installed in one hand piece and the second laser diodes 522 may be installed in another hand piece.

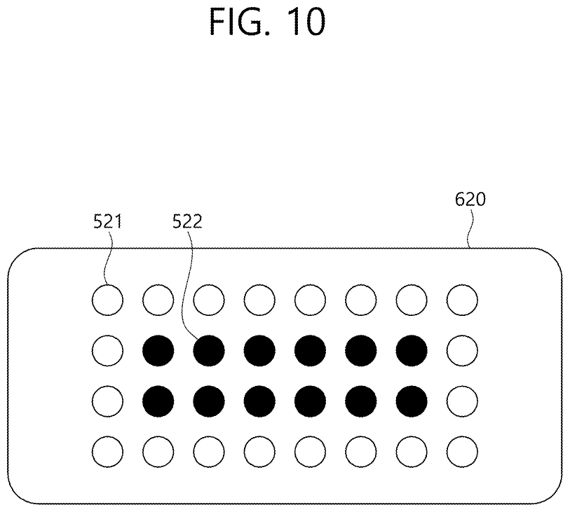

FIG. 10 is a diagram schematically illustrating a front end of a hand piece of a laser lipolysis apparatus according to a sixth embodiment of the present invention.

For convenience of explanation, the same reference numerals are used for elements similar to those from the above-described fifth embodiment, and descriptions of elements that the present exemplary embodiment has in common with the above-described fifth embodiment will be omitted.

As illustrated in FIG. 10, at a front end of a hand piece 620 of the laser lipolysis apparatus according to the sixth embodiment of the present invention, first laser diodes 521 and second laser diodes 522 are arranged in separate regions.

FIG. 10 illustrates an example in which the second laser diodes 522 are centered and the first laser diodes 521 are arranged to surround the second laser diode 522.

However, according to an embodiment, the arrangement relationship between the first laser diodes 521 and the second laser diodes 522 may change. For example, the first laser diodes 521 may be centered and the second laser diodes 522 may be arranged to surround the first laser diodes 521, or the first laser diodes 521 and the second diodes 522 may be arranged to be vertically or horizontally separated from each other.

Those of ordinary skill in the art to which the present disclosure pertains should understand that the present disclosure may be practiced in other specific forms without changing the technical idea or essential features thereof. Therefore, the embodiments described herein are illustrative in all aspects and should not be understood as limiting. The scope of the present disclosure is shown by the claims below rather than the detailed description given above, and all changes or modifications derived from the meaning and the scope of the claims and their equivalents should be interpreted as belonging to the scope of the present disclosure.

### Mode for the Invention

A laser lipolysis apparatus according to an embodiment of the present invention include a first light irradiation unit for emitting a laser of a first wavelength reaching an upper region of a subcutaneous fat layer, and a second light irradiation unit for emitting a laser of a second wavelength reaching a lower region of the subcutaneous fat layer.

A laser lipolysis procedure method according to an embodiment of the present invention includes irradiating skin of a patient with a laser of a first wavelength reaching an upper region of a subcutaneous fat layer, and irradiating the skin of the patient with a laser of a second wavelength reaching a lower region of the subcutaneous fat layer.

1. A laser lipolysis apparatus comprising:
a first light irradiation unit for emitting a laser of a first wavelength reaching an upper region of a subcutaneous fat layer; and
a second light irradiation unit for emitting a laser of a second wavelength reaching a lower region of the subcutaneous fat layer.

2. The laser lipolysis apparatus of claim 1, wherein the laser of the first wavelength and the laser of the second wavelength are irradiated toward a skin surface such that at least some of the lasers penetrate epidermis and dermis and reach the subcutaneous fat layer.

3. The laser lipolysis apparatus of claim 1, wherein the first wavelength is shorter than the second wavelength.

4. The laser lipolysis apparatus of claim 2, wherein the first wavelength is 635 nm and the second wavelength is 1064 nm.

5. The laser lipolysis apparatus of claim 1, wherein the laser of the first wavelength and the laser of the second wavelength are emitted alternately.

6. The laser lipolysis apparatus of claim 1, further comprising:
a first hand piece provided with the first light irradiation unit; and
a second hand piece provided with the second light irradiation unit.

7. The laser lipolysis apparatus of claim 1, further comprising a hand piece provided with the first light irradiation unit and the second light irradiation unit.

8. The laser lipolysis apparatus of claim 1, further comprises a fixed member for fixing at least one of the first light irradiation unit or the second light irradiation unit to a body of a patient.

9. The laser lipolysis apparatus of claim 8, wherein the first light irradiation unit and the second light irradiation unit are detachably installed in the fixed member.

10. The laser lipolysis apparatus of claim 8, wherein the fixed member is brought into contact tightly with the body of the patient while surrounding at least one of abdomen, a leg, or an arm of the patient.

11. A laser lipolysis procedure method comprising:
irradiating skin of a patient with a laser of a first wavelength reaching an upper region of a subcutaneous fat layer; and
irradiating the skin of the patient with a laser of a second wavelength reaching a lower region of the subcutaneous fat layer.

12. The laser lipolysis procedure method of claim 11, wherein the first wavelength is shorter than the second wavelength.

13. The laser lipolysis procedure method of claim 12, wherein the first wavelength is selected within a range equal to or greater than 630 nm and equal to or smaller than 640 nm, and the second wavelength is selected within a range equal to or greater than 1060 nm and equal to or smaller than 1070 nm.

14. The laser lipolysis procedure method of claim 11, wherein the laser of the first wavelength and the laser of the second wavelength are irradiated alternately.
